(19) United States
(12) Patent Application Publication
(10) Pub. No.: US 2025/0145552 A1
(43) Pub. Date: May 8, 2025

(54) PROCESS FOR REDUCTION OF CARBOXYLIC ACIDS WITH AMMONIA BORANE CATALYZED BY LEWIS ACID

(71) Applicant: Purdue Research Foundation, West Lafayette, IN (US)

(72) Inventors: Padinjaremadhom V. Ramachandran, West Lafayette, IN (US); Henry John Hamann, West Lafayette, IN (US)

(21) Appl. No.: 18/935,759

(22) Filed: Nov. 4, 2024

Related U.S. Application Data
(60) Provisional application No. 63/547,844, filed on Nov. 8, 2023.

(51) Int. Cl. C07C 29/147

(52) U.S. Cl. CPC C07C 29/147; C07C 2521/06

(57) ABSTRACT

A process for reducing a carboxylic acid to an alcohol using an amine-borane in the presence of a Lewis acid catalyst.

### CROSS-REFERENCE TO RELATED APPLICATIONS

This application claims priority to U.S. provisional patent application No. 63/547,844, which was filed Nov. 8, 2023, and which is hereby incorporated by reference in its entirety.

### TECHNICAL FIELD

The present disclosure relates to a process for reducing a carboxylic acid to an alcohol utilizing an amine-borane in the presence of a Lewis acid catalyst.

### BACKGROUND

This section introduces aspects that may help facilitate a better understanding of the disclosure. Accordingly, these statements are to be read in this light and are not to be construed as admissions about what is or is not prior art.

The significance of the direct reduction of carboxylic acids to alcohols stems from their versatility as building blocks for pharmaceutical and fine chemical applications and their natural abundance. The low electrophilicity and high oxidation state of the carbonyl carbon make a reduction of acids challenging by using reducing agents such as hydrides. The hazards associated with the air and moisture sensitivity of the reducing agents and their potentially explosive incompatibility with certain functional groups add to the difficulty. While borane-tetrahydrofuran (BTHF) is efficient in reducing both aromatic and aliphatic acids at room temperature (RT), borane dimethyl sulfide (BMS) is inefficient in reducing aromatic acids. The reduction of aromatic acids with BMS is purportedly improved by their reported conversion to the corresponding acyloxyborane with the addition of trimethyl borate (Lane et al., Journal of Organic Chemistry, 1974, 39, 3052-3054).

Air and moisture-stable amine-boranes can be considered a safe alternative for carboxylic acid reduction. However, the increased Lewis basicity of nitrogen relative to oxygen and sulfur renders them weak as reducing agents. Several amine-boranes, including pyridine-borane and trisubstituted boranes such as triethylamine-, N,N-diethylaniline-, and N-ethyl-N-isopropylaniline-borane complexes have been examined for the reduction of carboxylic acids. However, the disadvantages of using these amine-boranes are poor product yields, limited substrate scope, and their decomposition.

Hence, there is an unmet need for a process for the reduction of a carboxylic acid to an alcohol using a stable reagent and mild conditions that are applicable to both aromatic and aliphatic carboxylic acids and provide a better yield. It is an object of the present disclosure to provide such a process. This and other objects and advantages, as well as inventive features, will be apparent from the detailed description.

### SUMMARY

Provided is a process for reducing a carboxylic acid to an alcohol, which process comprises reacting a carboxylic acid with an amine borane in the presence of a Lewis acid catalyst and a solvent.

The amine borane used for the reduction is represented by formula (I):

RR′R″N→BH₃ (I)
wherein each R, R′, and R″ independently is alkyl, cycloalkyl, heteroalkyl, heterocycloalkyl, aryl, heteroaryl, alkylaryl, or arylalkyl, wherein each R, R′ and R″ is optionally independently substituted with at least one of halo, OH, OR, CONR, alkyl, cycloalkyl, heteroalkyl, heterocycloalkyl, aryl, heteroaryl, alkylaryl, and arylalkyl; or
R, R′ and R″ together, with the nitrogen atom to which they are attached, form a 4- to 8-membered heteroaryl ring through an alkylene group; or
R and R′ together, with the nitrogen atom to which they are attached, form a 4- to 8-membered heterocyclic ring, which contains one or more heteroatoms.

In some embodiments, the amine borane is:

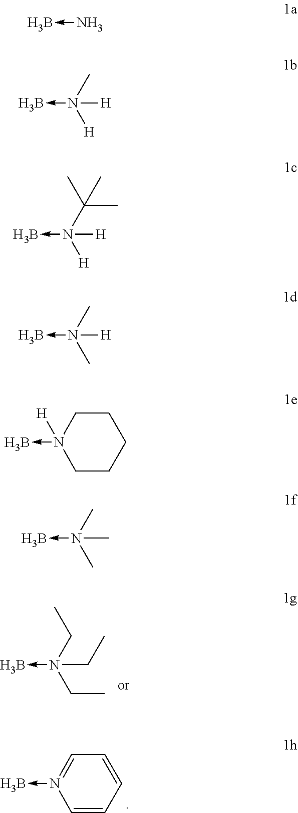

A desirable amine-borane is an ammonia borane (1a).

The carboxylic acid can be either an aliphatic carboxylic acid or an aromatic carboxylic acid. In some embodiments, the carboxylic acid is represented by formula (II):

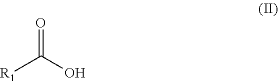

wherein R₁ is selected from the group consisting of alkyl, cycloalkyl, heteroalkyl, heterocycloalkyl, aryl, heteroaryl, alkylaryl, and arylalkyl, wherein R₁ is optionally substituted with at least one of halo, OH, OR₁, CONR₁, alkyl, cycloalkyl, heteroalkyl, heterocycloalkyl, aryl, heteroaryl, alkylaryl, and arylalkyl.

In some embodiments, the alcohol is represented by formula (III):

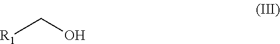

wherein R₁ is selected from the group consisting of alkyl, cycloalkyl, heteroalkyl, heterocycloalkyl, aryl, heteroaryl, alkylaryl, and arylalkyl, wherein R₁ is optionally substituted with at least one of halo, OH, OR₁, CONR₁, alkyl, cycloalkyl, heteroalkyl, heterocycloalkyl, aryl, heteroaryl, alkylaryl, and arylalkyl.

Any suitable Lewis acid, as is known in the art, can be used as a catalyst. Examples of Lewis acids include, but are not limited to, titanium tetrabromide (TiBr₄), hafnium tetrachloride (HfCl₄), boron trifluoride etherate (BF₃·OEt₂O), aluminium chloride (AlCl₃), bismuth chloride (BiCl₃), ferric chloride (FeCl₃), indium chloride (InCl₃), copper chloride (CuCl₂), zirconium chloride (ZrCl₄), zinc chloride (ZnCl₂), scandium triflate (Sc(OTf)₃), tin chloride (SnCl₂), tin tetrachloride (SnCl₄), and titanium tetrachloride (TiCl₄). In some embodiments, the Lewis acid is TiCl₄.

Lewis acid is used in a catalytic amount. The amount of Lewis acid used can be about 0.01 mol % to about 100 mol % relative to an amount of carboxylic acid. In some embodiments, the amount of Lewis acid used is about 0.01 mol % to about 20 mol % relative to the amount of carboxylic acid. Desirably, the amount is about 0.05 mol % to about 10 mol % relative to the amount of carboxylic acid.

In some embodiments, the amount of amine-borane used can be based on the amount of carboxylic acid used. The amount of amine-borane used can be from about 100 mol % to about 200 mol % of the amount of carboxylic acid. Desirably, the amount of amine-borane, based on the amount of carboxylic acid, is about 100 mol %.

In some embodiments, the catalyst is added to a solution of carboxylic acid prior to the addition of an amine-borane. The solvent used for the reduction process is an organic solvent. The organic solvent used can be selected from diethyl ether, tetrahydrofuran, dichloromethane, 2-methyl tetrahydrofuran, 1,2-dimethoxyethane, methyl tert-butyl ether, and acetonitrile. In some embodiments, the reduction is carried out at a temperature from about 0° C. to about 150° C.

In some embodiments, the process is carried out in a batch process or a continuous process.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The “arrow” in an amine-borane represents the coordinate covalent bond between nitrogen from NRR′R″ and Boron.

The terms “Lewis acid catalyst” and “catalyst” are used interchangeably.

Abbreviations used are:
NH₂Me—methylamine, NH₂tBu—tert-butylamine, NHMe₂—dimethylamine, NMe₃—trimethylamine, NEt₃—triethylamine, and tert—tertiary.

Reduction of carboxylic acids under mild conditions without reducing other functional groups is challenging. The reducing agents, such as borane-tetrahydrofuran and lithium aluminium hydride, are extremely reactive, require high pressure and temperature conditions, and the hydrogenation process often results in the formation of esters.

In view of the above, a mild process for direct reduction of a carboxylic acid to an alcohol that can be achieved at ambient conditions using an air and moisture-stable reagent is provided.

Provided is a process for reducing a carboxylic acid to an alcohol, which process comprises reacting a carboxylic acid with an amine borane in the presence of a Lewis acid catalyst and a solvent.

A carboxylic acid can be reduced directly to an alcohol using an amine borane. The amine borane can be represented by formula (I):

RR′R″N→BH₃ (I)
wherein each R, R′, and R″ independently is alkyl, cycloalkyl, heteroalkyl, heterocycloalkyl, aryl, heteroaryl, alkylaryl, or arylalkyl, wherein each R, R′, and R″ is optionally independently substituted with at least one of halo, OH, OR, CONR, alkyl, cycloalkyl, heteroalkyl, heterocycloalkyl, aryl, heteroaryl, alkylaryl, and arylalkyl; or
R, R′ and R″ together, with the nitrogen atom to which they are attached, form a 4- to 8-membered heteroaryl ring through an alkylene group; or
R and R′ together, with the nitrogen atom to which they are attached, form a 4- to 8-membered heterocyclic ring, which contains one or more heteroatoms.

In some embodiments, the amine borane is:

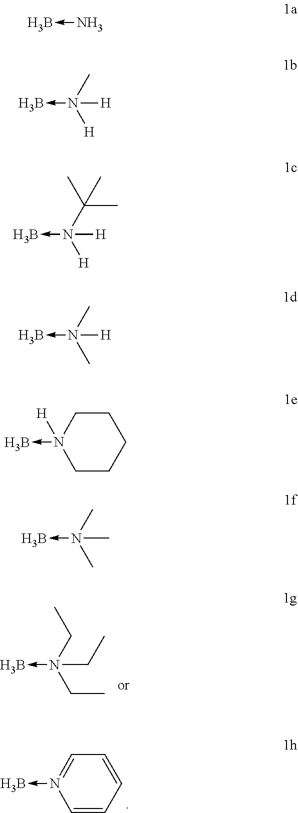

wherein, 1a is ammonia-borane, 1b is methylamine-borane, 1c is tert-butylamine-borane, 1d is dimethylamine-borane, 1e is piperidine-borane, 1f is tri-methylamine-borane, 1g is triethylamine-borane or 1h is pyridine-borane.

In some embodiments, the amine borane is ammonia borane (AB, 1a). Ammonia-borane can be used as a hydride source.

The carboxylic acid can be a mono-carboxylic acid or a dicarboxylic acid. In some embodiments, the carboxylic acid can be an aliphatic carboxylic acid or aromatic carboxylic acid, wherein the aromatic ring or aliphatic chain can be optionally substituted. The carboxylic acid can be represented by formula (II):

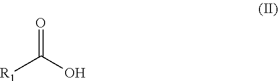

wherein R₁ is selected from the group consisting of alkyl, cycloalkyl, heteroalkyl, heterocycloalkyl, aryl, heteroaryl, alkylaryl, and arylalkyl, wherein R₁ is optionally substituted with at least one of halo, OH, OR₁, CONR₁, alkyl, cycloalkyl, heteroalkyl, heterocycloalkyl, aryl, heteroaryl, alkylaryl, and arylalkyl.

In some embodiments, the alcohol is represented by formula (III):

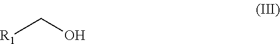

wherein R₁ is selected from the group consisting of alkyl, cycloalkyl, heteroalkyl, heterocycloalkyl, aryl, heteroaryl, alkylaryl, and arylalkyl, wherein R₁ is optionally substituted with at least one of halo, OH, OR₁, CONR₁, alkyl, cycloalkyl, heteroalkyl, heterocycloalkyl, aryl, heteroaryl, alkylaryl, and arylalkyl.

The alkyl can be C₁-C₆ alkyl. The cycloalkyl can be C₃-C₈ cycloalkyl. Lewis acid used can be any suitable Lewis acid as is known in the art. The Lewis acid can be selected from titanium tetrabromide (TiBr₄), hafnium tetrachloride (HfCl₄), boron trifluoride etherate (BF₃·OEt₂O), aluminium chloride (AlCl₃), bismuth chloride (BiCl₃), ferric chloride (FeCl₃), indium chloride (InCl₃), copper chloride (CuCl₂), zirconium chloride (ZrCl₄), zinc chloride (ZnCl₂), scandium triflate (Sc(OTf)₃), tin chloride (SnCl₂), tin tetrachloride (SnCl₄), and titanium tetrachloride (TiCl₄). In some embodiments, the Lewis acid is TiCl₄.

The Lewis acid is used in a catalytic amount. The amount of Lewis acid used can be about 0.01 mol % to about 100 mol % relative to the amount of carboxylic acid, such as about 0.01 mol % to 100 mol % or 0.01 mol % to about 100 mol % or 0.01 mol % to 100 mol % relative to the amount of carboxylic acid. In some embodiments, the amount of Lewis acid used is about 0.01 mol % to about 20 mol % relative to the amount of carboxylic acid, such as about 0.01 mol % to 20 mol % or 0.01 mol % to about 20 mol % or 0.01 mol % to 20 mol %. Desirably, the amount is about 0.05 mol % to about 10 mol % relative to the amount of carboxylic acid (such as 0.05 mol % to 10 mol % relative to the amount of carboxylic acid).

The amount of amine-borane used, based on the amount of carboxylic acid, can be from about 100 mol % to about 200 mol %, such as from about 100 mol % to 200 mol % or 100 mol % to about 200 mol % or 100 mol % to 200 mol %. In some embodiments, the amount of amine-borane used, based on the amount of carboxylic acid, is about 100 mol % (such as 100 mol %).

Any suitable organic solvent, as is known in the art, can be used for the reduction process. Examples of organic solvents include, but are not limited to, tetrahydrofuran (THF), dichloromethane (DCM), 1,2-dimethoxyethane (DME), acetonitrile (MeCN), diethyl ether (Et₂O), 2-methyl tetrahydrofuran, and methyl tert-butyl ether (MTBE). In some embodiments, the solvent is Et₂O.

The reduction can be carried out at a temperature from about 0° C. to about 150° C., such as from about 0° C. to 150° C. or from 0° C. to about 150° C. or from 0° C. to 150° C. In some embodiments, the reduction can be carried out at a temperature from about 10° C. to about 110° C., such as from about 10° C. to 110° C. or from 10° C. to about 110° C. or from 10° C. to 110° C. Desirably, the reduction is carried out at a temperature from about 20° C. to about 85° C. (such as from about 20° C. to 85° C. or from 20° C. to about 85° C.).

The reduction can be carried out at an ambient pressure. In some embodiments, the pressure can be in a range of about 0.1 bar to about 10 bar, such as about 0.1 bar to 10 bar or 0.1 bar to about 10 bar or 0.1 bar to 10 bar. Desirably, the pressure is about 0.5 bar to about 2.5 bar (such as 0.5 bar to 2.5 bar).

In some embodiments, the process comprises adding a Lewis acid catalyst to a carboxylic acid in a solvent prior to adding an amine-borane. The acidic site of the Lewis acid can coordinate with the carbonyl group of the carboxylic acid.

In some embodiments, the process of reduction comprises contacting a carboxylic acid, an amine borane (e.g., ammonia borane), and a Lewis acid catalyst (e.g., TiCl₄) in the presence of an organic solvent in a reaction vessel, which is a batch process. The reaction can also be carried out easily in a continuous process.

In some embodiments, the carboxylic acid is:

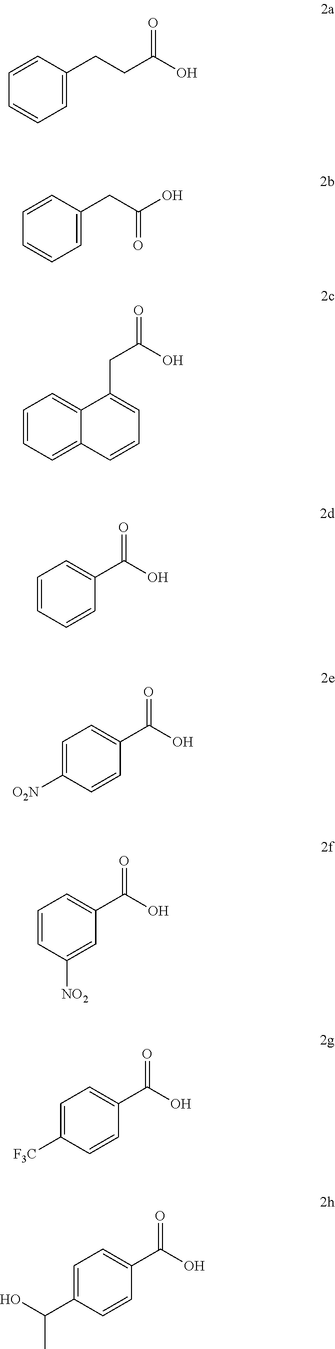

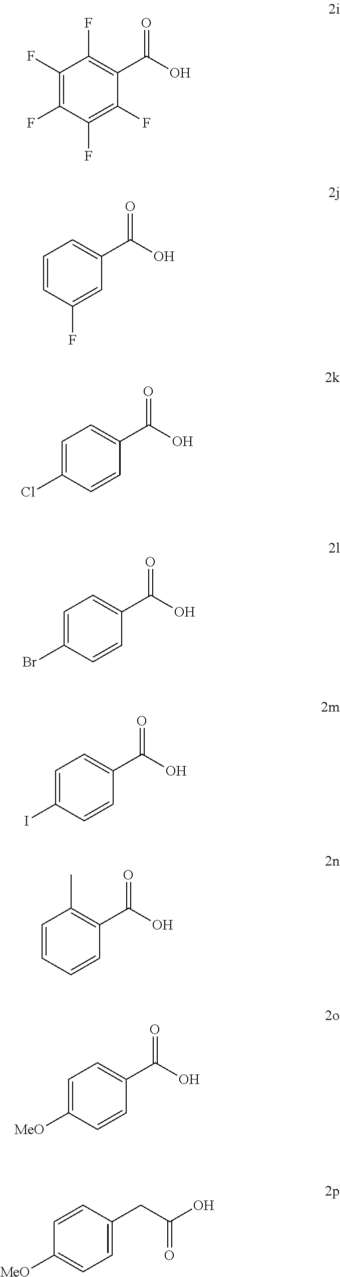

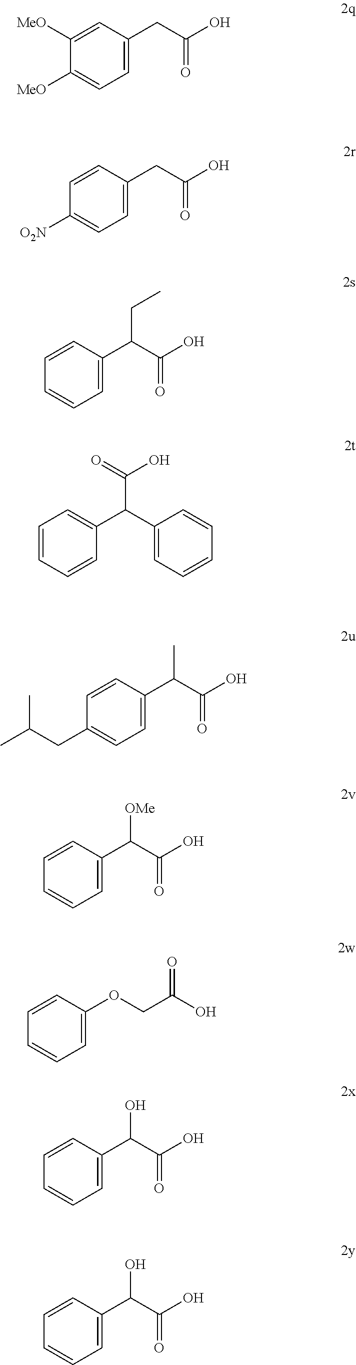

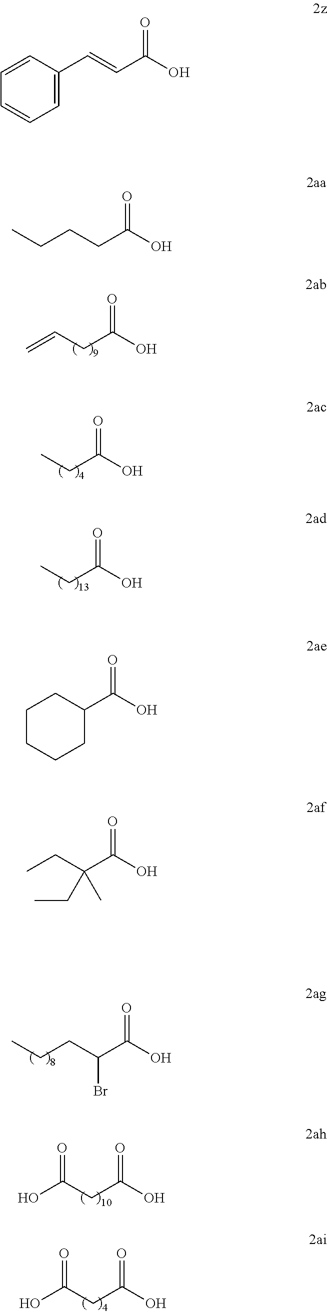

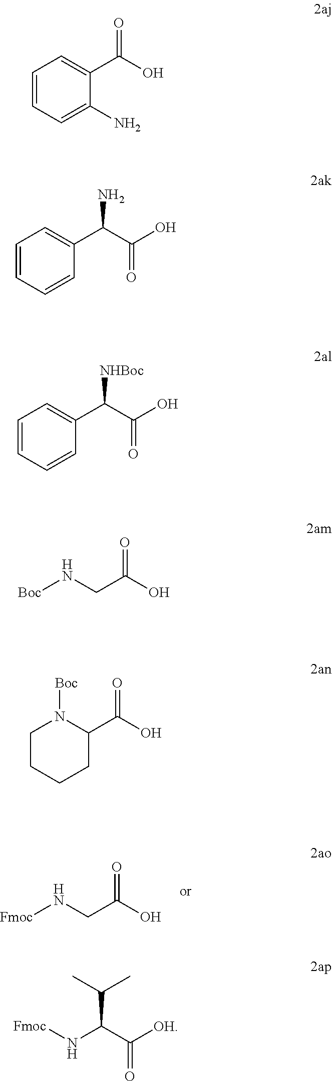

The reduction process of carboxylic acids was optimized: 3-phenylpropanoic acid (2a) was selected as the representative carboxylic acid for optimization and utilizing the same conditions as for the catalyzed reduction of ketones (Ramachandran, P. V. et al., J. Org. Chem. 2022, 87(19): 13259-13269), which is hereby specifically incorporated by reference for its teachings regarding the same. For example, the reduction was carried out using 1 equiv AB (1a), and 10 mol % TiCl₄ in diethyl ether (Et₂O) at RT. The product 3-phenylpropanol (3a) was obtained with a yield of 76% within 4 hours. Theoretically, the conversion of the carboxylic acid to alcohol would require only 3 hydride equivalents (1 equiv AB), including the hydride liberated as molecular hydrogen, following the reaction with a carboxylic acid. Experiments employing 1.5 and 2 equiv of AB yielded 84% and 91% of 3a, respectively. Accordingly, 2 equiv of AB (1a) were used for further optimization of the reaction (see Table 1). In comparison, the successful reduction of carboxylic acids with sodium borohydride (SBH) requires 110 mol % TiCl₄ and ˜13 equiv of hydride (3.3 molar equiv of SBH) at RT for 14 hours (Cho, B. T. et al., Synthetic Communication, 1985, 15, 917-924).

Table 1 summarizes the reaction optimization results. Examination of the catalyst loading revealed that 5 mol % of TiCl₄ provided only 31% conversion to 3a, and in the absence of TiCl₄, no reaction was observed even after 24 hours, establishing the necessity of the catalyst for the transformation. The effect of the solvent was explored by replacing Et₂O with THF, DCM, DME, MTBE or MeCN. Under similar conditions, all of these, with the exception of MTBE gave drastically lower yields of the desired 3a, confirming the suitability of Et₂O. A reaction in MTBE provided 60% yield of the alcohol (see Table 1, entry 10).

TABLE 1
Reaction optimization

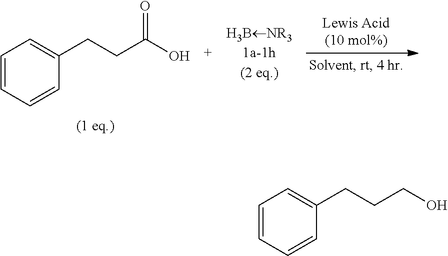 [SMILES: O=C(O)CCc1ccccc1 ; OCCCc1ccccc1]

Amine-boranes
H₃B←NH₃
1a

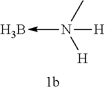

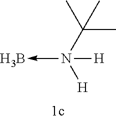

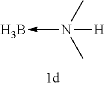

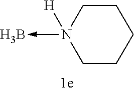

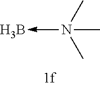

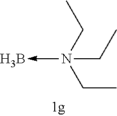

or

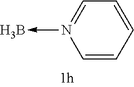

| entry | amine-borane | Lewis Acid^{a} | solvent | yield^{b} |
|---|---|---|---|---|
| 1 | 1a^{c} | TiCl₄ | Et₂O | 76 |
| 2 | 1a^{d} | TiCl₄ | Et₂O | 84 |
| 3 | 1a | TiCl₄ | Et₂O | 91 |
| 4 | 1a | TiCl₄^{e} | Et₂O | 31 |
| 5 | 1a | none | Et₂O | No |
| 6 | 1a | TiCl₄ | THF | 12 |
| 7 | 1a | TiCl₄ | DCM | 43 |
| 8 | 1a | TiCl₄ | DME | 22 |
| 9 | 1a | TiCl₄ | CH₃CN | 37 |
| 10 | 1a | TiCl₄ | MTBE | 60 |
| 11 | 1a | TiBr₄ | Et₂O | 66 |
| 12 | 1a | HfCl₄ | Et₂O | 40 |
| 13 | 1a | BF₃•OEt₂O | Et₂O | 35 |
| 14 | 1a | AlCl₃ | Et₂O | 25 |
| 15 | 1a | BiCl₃ | Et₂O | 15 |
| 16 | 1a | FeCl₃ | Et₂O | 3 |
| 17 | 1a | InCl₃ | Et₂O | 11 |
| 18 | 1a | CuCl₂ | Et₂O | 7 |
| 19 | 1a | ZrCl₄ | Et₂O | 6 |
| 20 | 1a | ZnCl₂ | Et₂O | 5 |
| 21 | 1a | Sc(OTf)₃ | Et₂O | 17 |
| 22 | 1a | SnCl₂ | Et₂O | 5 |
| 23 | 1a | SnCl₄ | Et₂O | 9 |
| 24 | 1b | TiCl₄ | Et₂O | 43 |
| 25 | 1c | TiCl₄ | Et₂O | 13 |
| 26 | 1d | TiCl₄ | Et₂O | 2 |
| 27 | 1e | TiCl₄ | Et₂O | 1 |
| 28 | 1f | TiCl₄ | Et₂O | 64 |
| 29 | 1g | TiCl₄ | Et₂O | 13 |
| 30 | 1h | TiCl₄ | Et₂O | 9 |

^{a}0.1 mol % TiCl₄
^{b}Isolated yield.
^{c}1 equiv of AB.
^{d}1.5 equiv of AB.
^{e}0.05% mol of TiCl₄

The Lewis acid catalysts, such as ZrCl₄ and HfCl₄ catalysts, proved inefficient. In addition, Lewis acids, such as, BF₃·Et₂O, AlCl₃, BiCl₃, FeCl₃, InCl₃, CuCl₂, ZnCl₂, Sc(OTf)₃, SnCl₂, and SnCl₄ were tested as catalysts. The catalysts, HfCl₄, BF₃·Et₂O, and AlCl₃, gave modest (25% to 40%) recovery of 3a. All other catalysts yielded very low or no product alcohol. TiCl₄ was exceptional, prompting the examination of TiBr₄, which provided only 66% of the alcohol.

Alternate hydride sources were examined by replacing 1a with a representative series of amine-boranes. The greatly decreased yields when using primary (1°) [methylamine-(1b) and tert-butylamine-(1c)], secondary (2°) [dimethylamine-(1d) and piperidine-(1e)], and tertiary (3°) [trimethylamine-(1f) and triethylamine-(1g)], and heteroaromatic [pyridine-borane (1h)]amine-boranes confirmed the superiority of 1a.

Carboxylic acids, which are reduced with ammonia-borane (1a, 2 equiv) in the presence of titanium tetrachloride (10 mol %) in Et₂O at RT, are summarized in Scheme 1. In the case of aliphatic carboxylic acid, the chain shortened and extended conjugation homologs, 2-phenylacetic acid (2b) and 2-(naphthalene-1-yl)acetic acid (2c), were converted to the corresponding alcohols 3b and 3c in 90% and 84% yields, respectively. A 100 mmol scale reaction of 2-phenylacetic (2b) performed using the standard conditions gave a slightly decreased yield of alcohol (3b). However, by increasing the TiCl₄ equivalency to 15%, 94.7% of 3b was recovered, demonstrating the scalability of the reaction. Aromatic carboxylic acids, such as benzoic acid (2d), furnished benzyl alcohol (3d) in a decreased (61%) yield within 4 hours. This result should be judged cognizant of the fact that other borane reagents, including diborane and borane-dimethylsulfide, are ineffective for the reduction of 2d. Extending the reaction to 12 hours had minimal effect. However, utilization of a full equivalent of TiCl₄ improved the yield of 3d to 89%.

Substituted benzoic acids containing electron-withdrawing 4- and 3-nitro (2e and 2f) and 4-trifluoromethyl (2g) groups gave the corresponding alcohols 3e, 3f, and 3g in 81%, 83% and 87%, respectively. Both the ketone and a carboxylic acid moiety of 4-acetylbenzoic acid (2h) were converted to the alcohol, providing the diol (3h) in 40% yield. Various halobenzoic acids (2i-2m) were each converted to the corresponding alcohols (3i-3m) in 83-90% yields, with none of the dehalogenation. The electron-donating methyl group of 2n did not inhibit the formation of alcohol (3n), isolated in 85% yield. However, the reaction of increased electron-donating 4-methoxybenzoic acid provided the corresponding alcohol (3o) in only 46% yield, even when the reaction was extended to 24 hours.

Scheme 1 illustrates the TiCl₄-catalyzed reduction of carboxylic acids with ammonia borane^{a}. Alcohols 3a-3ap were obtained by reducing carboxylic acids 2a-2ap (shown above), respectively.

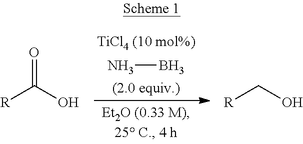

Alcohols from Carboxylic Acids

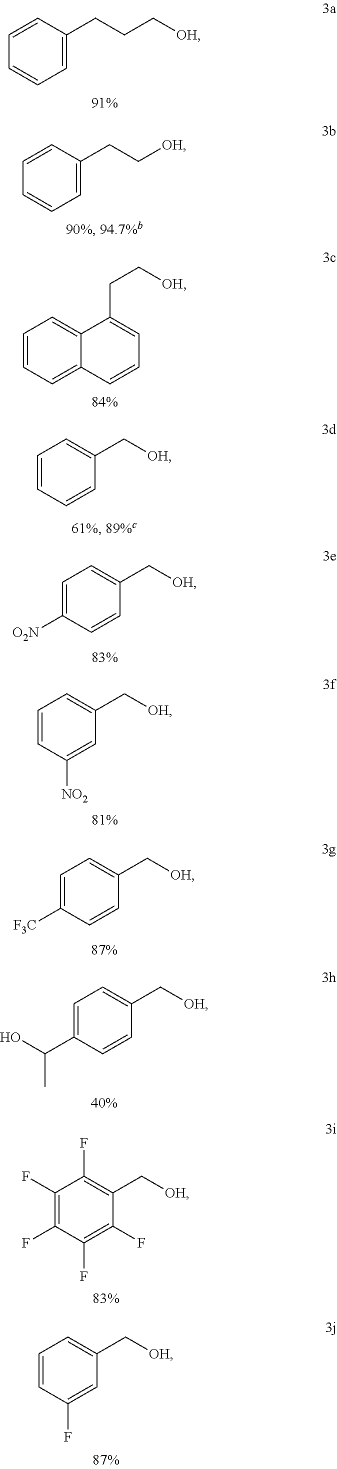

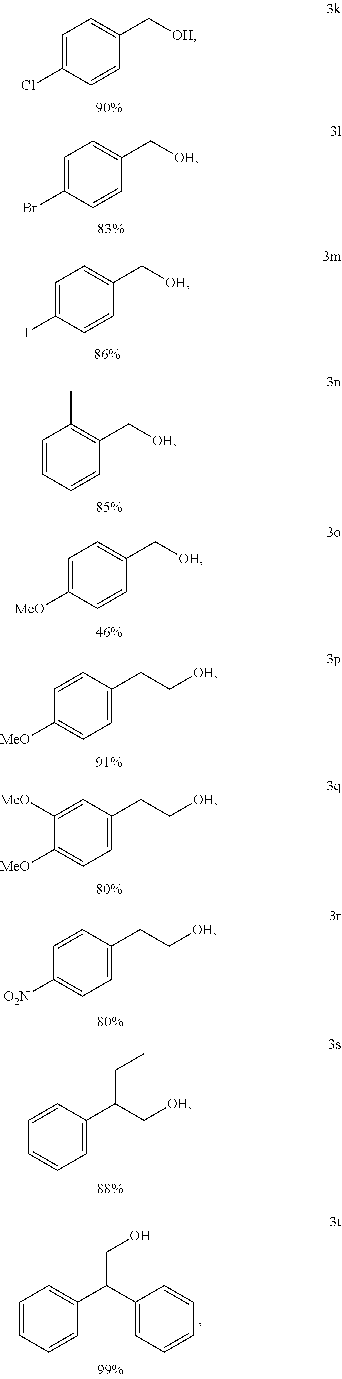

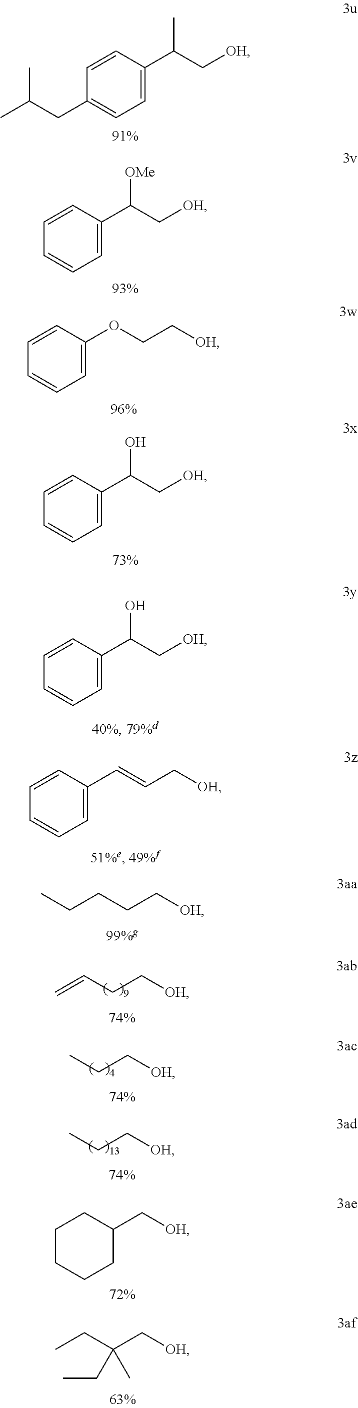

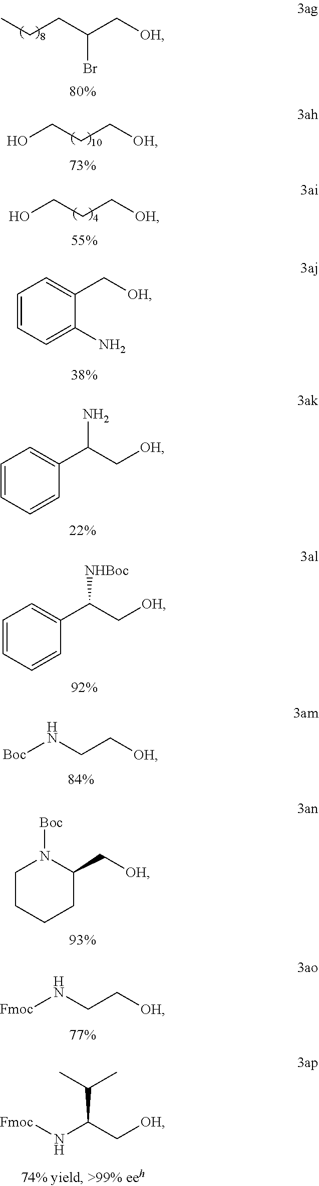

For 4-methoxyphenyl acetic acid (2p) or 3,4-dimethoxyphenylacetic acid (2q); alcohols (3p) and (3q) were obtained in 91% and 80% yields, respectively. The electron-withdrawing group in 4-nitrophenylacetic acid (2r) had no effect, and 2r was converted to the alcohol 3r in 80% yield. ^{a}Isolated yield shown, ^{b}Yield for 100 mmol scale reaction with 15% TiCl₄, ^{c}Extended reaction time for 12 hr., ^{d}3 eq. NH₃BH₃ and 0.5 eq. TiCl₄ used, ^{e} NMR ratio of Cinnamyl alcohol, ^{f}NMR ratio of 3-phenylpropan-1-ol, ^{g}From pent-2-enoic acid, NMR ratio, product not isolated due to low boiling point, ^{h}Determined by deprotection and conversion to menthyl chloroformate derivative.

Introduction of substitutions at the α-position of phenylacetic acid, including ethyl (2s), phenyl (2t), methyl (2u), and methoxy (2v) groups were well tolerated, providing alcohols 3s-3v in 88-99% yields. 2-Phenoxyacetic acid (2w) also provided a 96% yield of alcohol 3w. A slight decrease in yield was observed for mandelic acid (2x), when the diol (3x) was obtained in 73% yield. Similar to the reaction of (2h), another keto acid, phenyl glyoxylic acid (benzoylformic acid, 2y), was reduced to the diol (3y) in 40% by the conversion of the ketone and acid functionalities. Increasing the stoichiometry of the reagents to 3 eq. AB and 0.5 eq. TiCl₄, diol (3y) was obtained in 79% yield. This is contrary to what has been previously observed when using borane-THF in the absence of a catalyst, where the ketone moiety stayed intact during the reduction of (2y).

The reaction of α,β-unsaturated, (E)-cinnamic acid provided a mixture of 51% of enol 3z and 49% of the saturated alcohol resulting from hydrogenation. Decreasing the stoichiometry of 1a to 1.5 eq. gave a ratio of 30% of 3z to 42% of the saturated alcohol, with 28% of the starting material (2z) remaining unreacted. The α,β-unsaturated, aliphatic alcohol (E)-pent-2-enoic acid yielded only the saturated alcohol (3aa). However, the isolated double bond of undec-10-enoic acid (2ab) remained unaffected, providing the olefinic alcohol (3ab) in 74% yield. This is in contrast to systems where BH₃ is released, including NaBH₄/I₂, in which 1,11-undecanediol is an observed product, resulting from the hydroboration of the alkenyl moiety.

In extending the protocol to aliphatic acids, acyclic (2ac and 2ad), cyclic (2ae), tertiary (2af), and halogen-substituted (2ag) substrates were well tolerated, providing the alcohols (3ac-3ag) in 63% to 80% yield. In a prior procedure using 1g for the reduction of 2ac in a 1:1 ratio, at high temperature (80° C.) ˜45% of the ethyl and hexyl esters were obtained along with the desired alcohol. Aliphatic dicarboxylic acids 2ah and 2ai were converted to the diols (3ah) and (3ai) in 73% and 55% yields, respectively. The decreased yield of 3ai may be attributed to loss during work-up due to its water solubility.

Using aromatic and aliphatic amino acids, anthranilic acid (2aj) and phenylglycine (2ak), the corresponding amino alcohols (3aj) and (3ak) were obtained in 38% and 22% yields, respectively. The low yields were improved by using N-protected amino acids. Both t-butoxycarbonyl (Boc) and fluorenylmethoxycarbonyl (Fmoc) protecting groups were tolerated, where the N-Boc-protected (3al-3an) and N-Fmoc-protected (3ao and 3ap) amino alcohols were provided in 84%-93% and 74%-77% yields respectively. A 20 mmol scale reaction of 2am was performed using 15 mol % TiCl₄ in MTBE as a solvent to examine the potential for replacing Et₂O with the green solvent MTBE. The corresponding N-Boc-protected amino alcohol (3am) was provided in 89% isolated yield, confirming the suitability of MTBE as a potential replacement for Et₂O. Previous reductions of N-protected amino acids using LiAlH₄ and DIBAL-H were ineffective due to the consumption of the hydride via protecting group hydrolysis. The effect of the reduction on the stereochemistry of the product alcohols was determined using 3ap as a representative example. Deprotection and menthyl chloroformate derivatization of 3ap produced a material whose gas chromatogram, obtained using a chiral column (CP-Chirasil-Dex CB), indicated complete stereochemical retention.

The concurrent reduction of the ketone and carboxylic acid moieties in substrates 2 h and 2y prompted a series of competitive reactions to ascertain the chemoselectivity of the TiCl₄/NH₃BH₃ system (Scheme 2). We had already reported the selective reduction of a ketone in the presence of an acid moiety. A confirmatory competitive reaction of phenylacetic acid (2b) and phenyl-2-propanone (4a) revealed a ratio of 3:97 of the product alcohols (3b:5a) from 2b and 4a, respectively, using the reported reagent equiv (0.5 equiv of 1a) for ketone reductions. Using the conditions for acid reduction (2 equiv of 1a), this ratio increased only slightly to 20:80 of 3b:5a, demonstrating that the ketone is still preferentially reduced even with an excess reductant.

A reaction of TiCl₄/NH₃BH₃ with a 1:1 mixture of phenylacetic acid (2b) and its methyl ester (4b) yielded an alcohol (3b) to acid to ester ratio of 51:11:38. The ratio of 3b produced indicated that although 2b is more reactive, the reduction of 4b also has contributed to the observed quantity of 3b. On the basis of the detected residual acid and ester, the acid is reduced at a rate 3 times that of the ester. Conducting the competitive reaction with 1.5 and 1.0 equiv of NH₃BH₃ in the presence of 10% TiCl₄ resulted in ratios of 39:19:42 and 31:26:43, respectively. When the reduction of the acid (2b) was compared with the corresponding N-benzyl amide (4c), only the acid was consumed, producing 78% of 3b, while none of the amine from the potential reduction of 4c was observed, establishing the chemoselectivity for acid reduction. Similarly, a competitive reaction between acid (2a) and the corresponding nitrile, 2-phenylacetonitrile (4d), also revealed that the reduction process is selective for the acid. None of the amine from the potential reduction of the nitrile was observed.

Scheme 2: Competitive reactions of carboxylic acids.

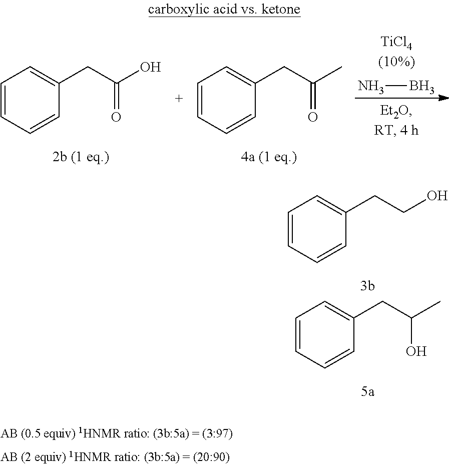

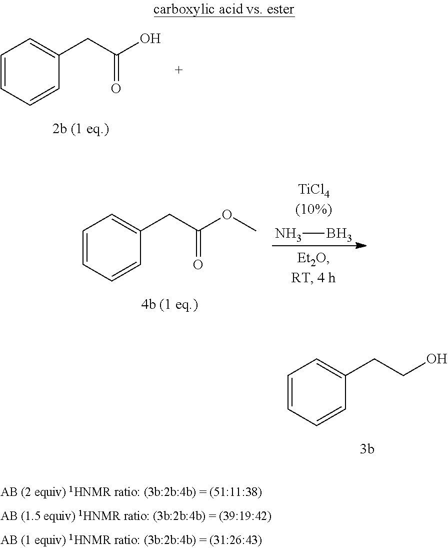

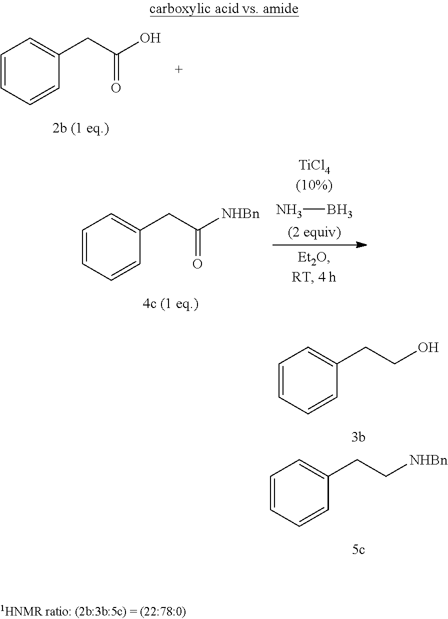

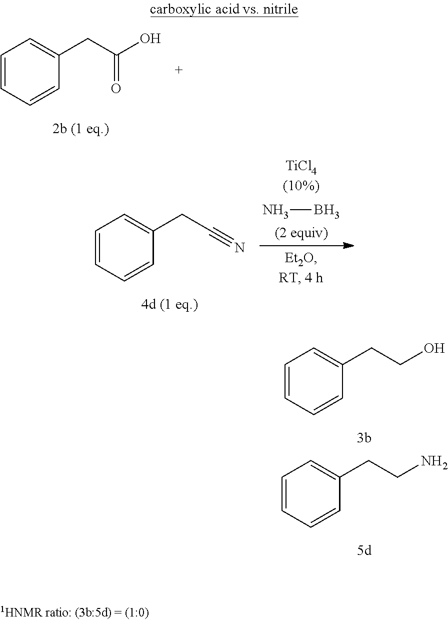

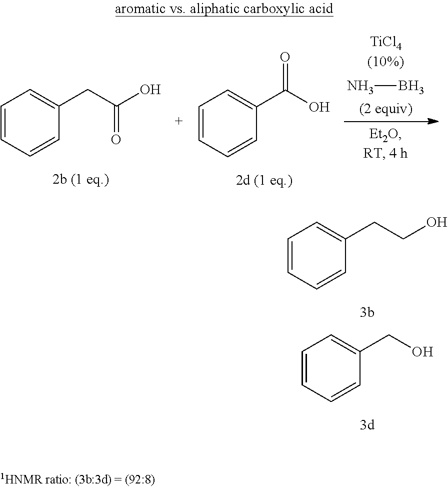

Competitive reactions between aromatic and aliphatic carboxylic acids were also selective, with aliphatic acids preferentially reduced. A reaction with 1:1 phenylacetic acid (2b) and benzoic acid (2d) provided the corresponding alcohols (3b) and (3d) in a ratio of 92:8.

### EXAMPLES

The following examples serve to illustrate the present disclosure. The examples are not intended to limit the scope of the claimed invention in any way.

### General Information:

All reagents and starting materials were purchased from Sigma-Aldrich, Oakwood, or Fisher Scientific. Carboxylic acid, amines, sodium bicarbonate, sodium borohydride, and amine-boranes 1b, 1d, and 1f were used as received. Anhydrous diethyl ether was prepared by distillation from sodium-benzophenone, and anhydrous dichloromethane was prepared by distillation from calcium hydride, both were stored under nitrogen atmosphere.

Thin layer chromatography (TLC) was performed on silica gel F60 plates and visualized under UV light or ceric ammonium molybdate solution. Column chromatography was carried out with Kieselgel silica gel 60 M. The products were confirmed by nuclear magnetic resonance (NMR) spectroscopy and measured in 6 values in parts per million (ppm).

¹H NMR spectra of reduction products were recorded from a Bruker 400 MHz spectrometer at ambient temperature and calibrated against the residual solvent peak of CDCl₃ (δ=7.26 ppm) as an internal standard. The ¹³C NMR spectra were reported at 101 MHz (297 K) and calibrated using CDCl₃ (δ=77.0 ppm) as an internal standard. Coupling constants (J) are given in hertz (Hz), and signal multiplicities are described of NMR data as s=singlet, d=doublet, t=triplet, dd=double doublet, dt=double triplet, q=quartet, p=pentet, m=multiplet, and br=broad. ¹¹B, ¹H (300 MHz), and ¹³C NMR (75 MHz) spectra of synthesized amine-boranes were recorded at room temperature on a Varian INOVA or MERCURY 300 MHz NMR instrument. ¹¹B NMR spectra were recorded at 96 MHz, and chemical shifts were reported relative to the external standard, BF3:OEt2 (δ=0 ppm).

### Procedure for the Preparation of Ammonia-Borane (1a)

H₃B—NH₃

Sodium borohydride (NaBH₄) (18.91 g, 500 mmol, 1 eq.) and powdered ammonia sulfate ((NH₄)₂SO₄) (66.07 g, 500 mmol, 1 eq.) were transferred to a dry 2 L round bottom flask containing a large magnetic stir bar. The flask was then cooled using an ice-water bath, followed by the addition of 495 mL of reagent-grade tetrahydrofuran. With vigorous stirring, 4.5 mL of water was then added dropwise over a period of 5 minutes to limit frothing. Once all water was added, the flask was moved to room temperature and stirred vigorously. The reaction was monitored using ¹¹B NMR until completion (2-4 hours) as judged by the absence of sodium borohydride peaks in the ¹¹B NMR. (Prior to running the ¹¹B NMR experiments, a drop of dimethyl sulfoxide was added to the aliquot to solubilize any sodium borohydride present.) Once complete, the reaction mixture was filtered through celite, and the filter cake was thoroughly rinsed with tetrahydrofuran. The combined filtrates were condensed via rotary evaporation following by drying in vacuo for 12 hours to yield ammonia-borane (1a). (White solid, 9.72 g, 63%).

¹H NMR (300 MHz, Tetrahydrofuran-d8) δ 4.26-3.69 (m, 3H), 2.08-0.80 (m, 3H). B NMR (96 MHz, Tetrahydrofuran-d8) 6-22.04 (q, J=95.6 Hz).

### Procedure for the Preparation of Amine-Boranes by Bicarbonate-Mediated Synthesis

Sodium borohydride (1.51 g, 2 eq., 40 mmol) and powdered sodium bicarbonate (6.72 g, 4 eq., 80 mmol) were transferred to a 100 mL dry round bottom flask, charged with a magnetic stir-bar. The corresponding amine (1 eq., 20 mmol) was charged into the reaction flask, followed by addition of reagent-grade tetrahydrofuran (20 mL) at room temperature. Under vigorous stirring, water (0.36 mL, 4 eq., 80 mmol) was added drop-wise to prevent excessive frothing. Reaction progress was monitored by ¹¹B NMR spectroscopy (Note: A drop of anhydrous DMSO was added to the reaction aliquot before running the ¹¹B NMR experiment to solubilize NaBH₄). Upon completion of the reaction (4-48 hours, as determined by ¹¹B NMR), the reaction contents were filtered through sodium sulfate and celite, and the solid residue was washed with THF. Removal of the solvent in vacuo from the filtrate yielded the corresponding amine-borane (1c, 1e, 1g, 1h). Residual solvent was removed by placing under high-vacuum for ˜12 hours.

### Preparation and Characterization of Ammonia-Borane and Amine-Boranes

### t-Butylamine-Borane (1c)

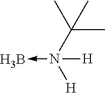

The compound was prepared as described in the procedure for the preparation of amine-boranes by bicarbonate mediated synthesis; (White solid, 1.62 g, 93%); ¹H NMR (300 MHz, CDCl₃) δ 1.30 (s, 9H); ¹³C{H} NMR (75 MHz, CDCl₃) δ 53.3, 28.1; ¹¹B NMR (96 MHz, CDCl₃) δ −23.22 (q, J=96.8, 96.2 Hz, 3H).

### Piperidine-borane (1e)

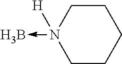

The compound was prepared as described in the procedure for the preparation of amine-boranes by bicarbonate mediated synthesis; (White solid, 1.94 g, 98%); ¹H NMR (300 MHz, CDCl₃) δ 3.90 (s, 1H), 3.17 (d, J=13.4 Hz, 2H), 2.42 (q, J=13.7, 12.5 Hz, 2H), 1.71 (d, J=10.9 Hz, 3H), 1.60-1.18 (m, 4H); ¹³C{H} NMR (75 MHz, CDCl₃) δ 53.3, 25.3, 22.6; ¹¹B NMR (96 MHz, CDCl₃) δ −14.64 (d, J=95.9 Hz, 3H).

### Triethylamine-borane (1g)

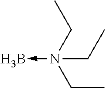

The compound was prepared as described in the procedure for the preparation of amine-boranes by bicarbonate mediated synthesis; (Colorless liquid, 2.17 g, 94%); ¹H NMR (300 MHz, CDCl₃) δ 2.78 (q, J=7.3 Hz, 6H), 1.19 (t, J=7.2 Hz, 9H); ¹³C{H} NMR (75 MHz, CDCl₃) δ 52.4, 8.8; ¹¹B NMR (96 MHz, CDCl₃) δ −13.94 (q, J=96.9 Hz, 3H).

### Pyridine-borane (1h)

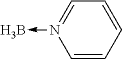

The compound was prepared as described in the procedure for the preparation of amine-boranes by bicarbonate mediated synthesis; (Colorless liquid, 1.83 g, 99%); ¹H NMR (300 MHz, CDCl₃) δ 8.49 (d, J=5.7 Hz, 2H), 7.89 (t, J=7.8 Hz, 1H), 7.46 (t, J=6.8 Hz, 2H), 3.14-1.96 (q, J=93.3 Hz, 3H); ¹³C{H} NMR (75 MHz, CDCl₃) δ 147.2, 139.3, 125.4; ¹¹B NMR (96 MHz, CDCl₃) δ −12.59 (q, J=97.4 Hz, 3H).

### General Reaction Optimization:

Reduction of 3-phenylpropanoic acid by amines-boranes in the presences of Lewis acids

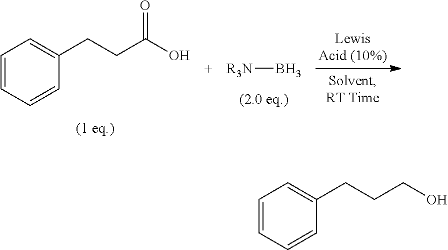

A 50 mL oven dried round bottom flask was charged with 3-phenylpropanoic acid (1 mmol, 1 eq) and a magnetic stirring bar. The flask was sealed using a rubber septum. After purging the flask with nitrogen, dry solvent (3 mL) was added, and the solution was cooled at 0° C. with an ice bath. Subsequently, the Lewis acid (0.1 mmol, 0.1 eq) was added to the solution, dropwise via syringe if a liquid or by temporarily removing the septum if a solid. The septum was then carefully open and solid amine-borane (2.0 mmol, 2.0 eq) was added slowly to the reaction mixture. Upon complete addition, the reaction flask was again sealed with a septum. After stirring at 0° C. for 1 minute, the reaction mixture was allowed to warm up to room temperature, stirred and monitored by TLC until completion. On completion of the reaction, the crude mixture was brought to 0° C. using an ice bath and quenched by slow addition of cold 1 M HCl, then transferred to a separatory funnel and extracted with diethyl ether (2×3 mL). The combined organic layers were washed with 1 M NaOH (1×3 mL) and then with brine (1×3 mL), dried over anhydrous sodium sulfate, filtered through cotton, and concentrated under rotary evaporation. The remaining solvent was removed by applying high vacuum for 30 minutes. The results of the optimization experiments are shown in Table 1.

### General Procedure for the Reduction of Carboxylic Acids (1 Mmol Scale)

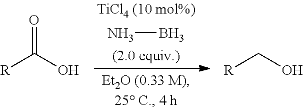

A 50 mL oven dried round bottom flask was charged with carboxylic acid (1 mmol, 1 eq) and a magnetic stir bar. The flask was sealed using a rubber septum. After purging the flask with nitrogen, distilled diethyl ether (3 mL) was added, and the solution was cooled at 0° C. with an ice bath. Subsequently, titanium tetrachloride (TiCl₄) (0.1 mmol, 0.1 eq) was added dropwise to the solution through the septum using a syringe. The septum was then carefully open and solid ammonia borane (NH₃·BH₃) (2.0 mmol, 2.0 eq) was added slowly to the reaction mixture. Upon complete addition, the reaction flask was again sealed with a septum. After stirring at 0° C. for 1 minute, the reaction mixture was allowed to warm up to room temperature, stirred and monitored by TLC until completion. On completion of the reaction, the crude mixture was brought to 0° C. and quenched by slow addition of cold 1 M HCl, then transferred to a separatory funnel and extracted with diethyl ether (2×3 mL). The combined organic layers were washed with 1 M NaOH (1×3 mL) and then with brine (1×3 mL), dried over anhydrous sodium sulfate, filtered through cotton, and concentrated under rotary evaporation and the remain solvent was removed by applying high vacuum for 30 minutes. Column chromatography was performed only if necessary to further purify the product using a hexanes:ethyl acetate (v/v=90:10) solvent system to afford the desired product.

### Procedure for the Reduction of Phenylacetic Acid (100 Mmol Scale)

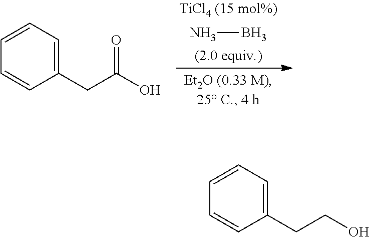

A 500 mL oven dried round bottom flask was charged with phenylacetic acid (13.615 g, 100 mmol, 1 eq) and a magnetic stir bar. The flask was sealed using a rubber septum, and connected to an indirect nitrogen line. After purging the flask with nitrogen, distilled diethyl ether (300 mL) was added via cannulation from a sealed graduated cylinder, and the solution was cooled at 0° C. with an ice bath. Subsequently, titanium tetrachloride (TiCl₄) (1.65 mL, 15 mmol, 0.15 eq) was added dropwise to the solution through the septum using a syringe. The septum was then carefully open, and solid ammonia borane (NH₃·BH₃) (6.173 g, 200 mmol, 2.0 eq) was added slowly in portions to the reaction mixture to prevent excessive foaming. Upon complete addition, the reaction flask was again sealed with a septum and connected to an indirect nitrogen line. After stirring at 0° C. for 1 minute, the reaction mixture was allowed to warm up to room temperature, stirred, and monitored by TLC until completion. On completion of the reaction, the crude mixture was brought to 0° C. with an ice bath and quenched by slow addition of cold 1 M HCl until the solution became clear, then transferred to a separatory funnel and extracted with diethyl ether (3×80 mL). The combined organic layers were washed with 1 M NaOH (1×50 mL) and then with brine (1×50 mL), dried over anhydrous sodium sulfate, filtered through cotton, and concentrated under rotary evaporation. The remaining solvent was removed by applying a high vacuum for overnight to yield a clear liquid (11.57 g, 94.7%).

### Preparation and Characterization of Alcohols from Carboxylic Acids

### 3-Phenylpropan-1-ol (3a)

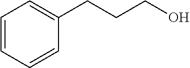

The compound was prepared as described in the general procedure (colorless oil, mass=124 mg, 91% yield); ¹H NMR (400 MHz, CDCl₃) δ 7.33-7.23 (m, 2H), 7.24-7.15 (m, 3H), 3.68 (t, J=6.4 Hz, 2H), 2.76-2.67 (m, 2H), 1.96-1.84 (m, 2H), 1.49 (s, 1H). ¹³C{H} NMR (101 MHz, CDCl₃) δ 141.7, 128.3, 128.3, 125.8, 62.2, 34.1, 32.0.

### 2-Phenylethan-1-ol (3b)

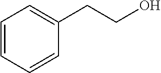

The compound was prepared as described in the general procedure (colorless oil, mass=110 mg, 90% yield); ¹H NMR (400 MHz, CDCl₃) δ 7.35-7.28 (m, 2H), −7.21 (m, 3H), 3.86 (t, J=6.5 Hz, 2H), 2.87 (t, J=6.6 Hz, 2H), 1.53 (s, 1H). ¹³C{H} NMR (101 MHz, CDCl₃) δ 138.4, 129.0, 128.5, 126.4, 63.6, 39.1.

### 2-(Naphthalen-1-yl)ethan-1-ol (3c)

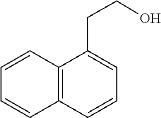 [SMILES: OCCc1cccc2ccccc12]

The compound was prepared as described in the general procedure (white solid, mass=145 mg, 84% yield); ¹H NMR (400 MHz, CDCl₃) δ 8.06 (d, J=8.1 Hz, 1H), 7.87 (d, J=8.4 Hz, 1H), 7.76 (d, J=7.9 Hz, 1H), 7.57-7.45 (m, 2H), 7.47-7.36 (m, 2H), 4.00 (t, J=6.4 Hz, 2H), 3.36 (t, J=6.7 Hz, 2H), 1.47 (s, 1H). ¹³C{H} NMR (101 MHz, CDCl₃) δ 134.3, 133.9, 132.0, 128.8, 127.3, 127.1, 126.0, 125.6, 125.4, 123.5, 63.0, 36.1.

### Benzyl Alcohol (3d)

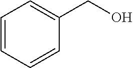

The compound was prepared as described in the general procedure (colorless oil, mass=96 mg, 89% yield); ¹H NMR (400 MHz, CDCl₃) δ 7.38-7.27 (m, 5H), 4.68 (s, 2H), 1.88 (s, 1H). ¹³C{H}NMR (101 MHz, CDCl₃) δ 140.8, 128.5, 127.6, 126.9, 65.3.

### (4-Nitrophenyl)methanol (3e)

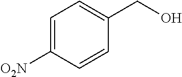 [SMILES: O=[N+]([O-])c1ccc(CO)cc1]

The compound was prepared as described in the general procedure (yellow solid, mass=127 mg, 83% yield); ¹H NMR (400 MHz, CDCl₃) δ 8.21 (d, J=8.7 Hz, 2H), 7.53 (d, J=9.0 Hz, 2H), 4.83 (s, 2H), 1.93 (s, 1H). ¹³C{H} NMR (101 MHz, CDCl₃) δ 148.0, 126.9, 123.6, 63.9.

### (3-Nitrophenyl)methanol (3f)

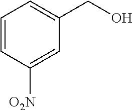 [SMILES: O=[N+]([O-])c1cccc(CO)c1]

The compound was prepared as described in the general procedure (light yellow solid, mass=124 mg, 81% yield); ¹H NMR (400 MHz, CDCl₃) δ 8.16 (p, J=2.6 Hz, 1H), 8.10-8.01 (m, 1H), 7.68-7.60 (m, 1H), 7.52-7.42 (m, 1H), 4.76 (s, 2H), 2.86 (s, 1H). ¹³C{H} NMR (101 MHz, CDCl₃) δ 148.2, 142.9, 132.6, 129.3, 122.3, 121.3, 63.7.

### 4-(Trifluoromethyl)-Benzyl alcohol (3g)

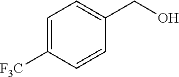 [SMILES: OCc1ccc(C(F)(F)F)cc1]

The compound was prepared as described in the general procedure (colorless oil, mass=153 mg, 87% yield); ¹H NMR (400 MHz, CDCl₃) δ 7.62 (d, J=7.8 Hz, 2H), 7.48 (d, J=7.9 Hz, 2H), 4.77 (s, 2H), 1.76 (s, 1H). ¹³C{H} NMR (101 MHz, CDCl₃) δ 144.6, 129.6, 126.7, 125.4, 125.3, 122.7, 64.3. ¹⁹F{H} NMR (376 MHz, CDCl₃) δ −64.02.

### 1-(4-(hydroxymethyl)phenyl)ethan-1-ol (3h)

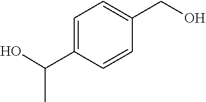

The compound was prepared as described in the general procedure (white solid, mass=61 mg, 40% yield); ¹H NMR (400 MHz, CDCl₃) δ 7.36-7.29 (m, 4H), 4.87 (q, J=6.4 Hz, 1H), 4.64 (s, 2H), 2.10 (s, 2H), 1.47 (d, J=6.4 Hz, 3H). ¹³C{H} NMR (101 MHz, CDCl₃) δ 145.1, 140.0, 128.0, 127.1, 125.5, 70.0, 64.9, 25.1.

### Pentafluorobenzyl Alcohol (3i)

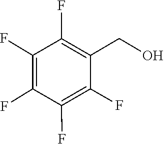 [SMILES: OCc1c(F)c(F)c(F)c(F)c1F]

The compound was prepared as described in the general procedure (white solid, mass=164 mg, 83% yield); ¹H NMR (400 MHz, CDCl₃) δ 4.80 (s, 2H), 2.04 (s, 1H). ¹³C{H} NMR (101 MHz, CDCl₃) δ δ 147.3-143.4 (m), 142.7-139.5 (m), 139.0-135.7 (m), 113.2 (t, J_{C-F}=17.1 Hz), 51.8. ¹⁹F{H} NMR (376 MHz, CDCl₃) δ −145.96, −155.62, −163.36.

### (3-Fluorophenyl)methanol (3j)

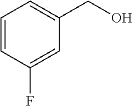

The compound was prepared as described in the general procedure (colorless oil, mass=110 mg, 87% yield); ¹H NMR (400 MHz, CDCl₃) δ 7.36-7.26 (m, 1H), 7.15-7.04 (m, 2H), 6.97 (td, J=8.5, 2.6 Hz, 1H), 4.68 (s, 2H), 1.93 (s, 1H). ¹³C{H} NMR (101 MHz, CDCl₃) δ 6 162.9 (d, J_{C-F}=245.6 Hz), 143.3 (d, J_{C-F}=7.2 Hz), 129.9 (d, J_{C-F}=8.2 Hz), 122.1 (d, J_{C-F}=2.7 Hz), 114.2 (d, J_{C-F}=21.2 Hz), 113.5 (d, J_{C-F}=21.6 Hz), 64.3. ¹⁹F{H} NMR (376 MHz, CDCl₃) δ −114.57.

### 2(4-Chlorophenyl)methanol (3k)

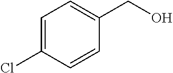

The compound was prepared as described in the general procedure (white solid, mass=128 mg, 90% yield); ¹H NMR (400 MHz, CDCl₃) δ 7.32 (d, J=8.6 Hz, 2H), 7.28 (d, J=8.6 Hz, 2H), 4.65 (s, 2H), 1.87 (s, 1H). ¹³C{H} NMR (101 MHz, CDCl₃) δ 139.2, 133.3, 128.6, 128.2, 64.4.

### (4-Bromophenyl)methanol (3l)

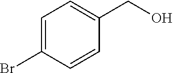

The compound was prepared as described in the general procedure (white solid, mass=155 mg, 83% yield); ¹H NMR (400 MHz, CDCl₃) δ 7.47 (d, J=8.4 Hz, 2H), 7.22 (d, J=8.7 Hz, 2H), 4.62 (s, 2H), 1.92 (s, 1H). ¹³C{H} NMR (101 MHz, CDCl₃) δ 139.7, 131.5, 128.5, 121.3, 64.4.

### (4-Iodophenyl)methanol (3m)

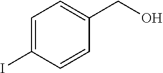

The compound was prepared as described in the general procedure (white solid, mass=201 mg, 86% yield); ¹H NMR (400 MHz, CDCl₃) δ 7.67 (d, J=8.3 Hz, 2H), 7.09 (d, J=8.3 Hz, 2H), 4.61 (s, 2H), 1.99 (s, 1H). ¹³C{H} NMR (101 MHz, CDCl₃) δ 140.3, 137.8, 137.5, 128.7, 92.9, 64.5.

### 2-Methylbenzyl alcohol (3n)

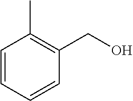

The compound was prepared as described in the general procedure (white solid, mass=104 mg, 85% yield); ¹H NMR (400 MHz, CDCl₃) δ 7.34 (d, J=3.9 Hz, 1H), 7.27-7.15 (m, 3H), 4.68 (s, 2H), 2.36 (s, 3H), 1.95 (s, 1H). ¹³C{H} NMR (101 MHz, CDCl₃) δ 138.6, 136.0, 130.3, 127.7, 127.4, 126.0, 63.3, 18.6.

### (4-Methoxyphenyl)methanol (3o)

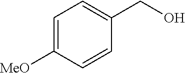

The compound was prepared as described in the general procedure (colorless oil, mass=64 mg, 46%; ¹H NMR (400 MHz, CDCl₃) δ 7.29 (d, J=8.8 Hz, 2H), 6.89 (d, J=8.7 Hz, 2H), 4.62 (s, 2H), 3.81 (s, 3H), 1.61 (s, 1H). ¹³C{H} NMR (101 MHz, CDCl₃) δ 159.1, 133.0, 128.6, 113.9, 64.9, 55.2.

### 2-(4-Methoxyphenyl)ethanol (3p)

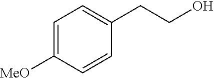 [SMILES: COc1ccc(CCO)cc1]

The compound was prepared as described in the general procedure (colorless oil, mass=138 mg, 91% yield; ¹H NMR (400 MHz, CDCl₃) δ 7.14 (d, J=8.7 Hz, 2H), 6.86 (d, J=8.6 Hz, 2H), 3.82 (t, J=6.6 Hz, 2H), 3.79 (s, 3H), 2.81 (t, J=6.6 Hz, 2H), 1.55 (s, 1H). ¹³C{H} NMR (101 MHz, CDCl₃) δ 158.2, 130.3, 129.9, 113.9, 63.7, 55.2, 38.2.

### 3,4-Dimethoxy-Benzyl ethanol (3q)

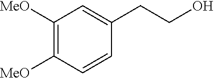 [SMILES: COc1ccc(CCO)cc1OC]

The compound was prepared as described in the general procedure (colorless oil, mass=146 mg, 80% yield); ¹H NMR (400 MHz, CDCl₃) δ 6.82 (d, J=7.9 Hz, 1H), 6.80-6.72 (m, 2H), 3.87 (s, 3H), 3.85 (s, 3H), 3.83 (t, J=6.5 Hz, 2H), 2.81 (t, J=6.5 Hz, 2H), 1.53 (s, 1H). ¹³C{H} NMR (101 MHz, CDCl₃) δ 148.9, 147.6, 130.9, 120.8, 112.1, 111.3, 63.6, 55.8, 55.8, 38.6.

### 2-(4-Nitrophenyl)ethanol (3r)

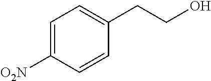 [SMILES: O=[N+]([O-])c1ccc(CCO)cc1]

The compound was prepared as described in the general procedure (yellow solid, mass=134 mg, 80% yield); ¹H NMR (400 MHz, CDCl₃) δ 8.16 (d, J=8.2 Hz, 2H), 7.40 (d, J=8.9 Hz, 2H), 3.92 (t, J=6.4 Hz, 2H), 2.97 (t, J=6.4 Hz, 2H), 1.60 (s, 1H). ¹³C{H} NMR (101 MHz, CDCl₃) δ 146.7, 129.8, 123.6, 62.8, 38.8.

### 2-Phenylbutan-1-ol (3s)

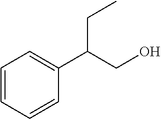

The compound was prepared as described in the general procedure (colorless oil, mass=132 mg, 88% yield); ¹H NMR (400 MHz, CDCl₃) δ 7.37-7.29 (m, 2H), 7.28-7.17 (m, 3H), 3.75 (qd, J=10.8, 6.8 Hz, 2H), 2.75-2.63 (m, 1H), 1.83-1.70 (m, 1H), 1.64-1.52 (m, 1H), 1.41 (s, 1H), 0.84 (t, J=7.4 Hz, 3H). ¹³C{H} NMR (101 MHz, CDCl₃) δ 142.2, 128.5, 128.0, 126.6, 67.2, 50.4, 24.9, 11.9.

### 2,2-Diphenyl ethanol (3t)

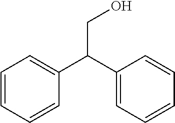 [SMILES: OCC(c1ccccc1)c1ccccc1]

The compound was prepared as described in the general procedure (colorless oil, mass=196 mg, 99% yield); ¹H NMR (400 MHz, CDCl₃) δ 7.37-7.19 (m, 10H), 4.25-4.14 (m, 3H), 1.50 (s, 1H). ¹³C{H} NMR (101 MHz, CDCl₃) δ 141.3, 128.6, 128.2, 126.7, 66.1, 53.6.

### 2-(p-isobutyl)propan-1-ol (3u)

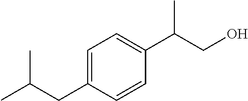

The compound was prepared as described in the general procedure (colorless oil, mass=175 mg, 91% yield); ¹H NMR (400 MHz, CDCl₃) δ 7.18-7.07 (m, 4H), 3.69 (d, J=6.8 Hz, 2H), 2.92 (q, J=7.0 Hz, 1H), 2.45 (d, J=7.1 Hz, 2H), 1.86 (dq, J=13.5, 6.8 Hz, 1H), 1.34 (s, 1H), 1.27 (d, J=7.0 Hz, 3H), 0.91 (d, J=6.6 Hz, 6H). ¹³C{H} NMR (101 MHz, CDCl₃) δ 140.6, 140.0, 129.3, 127.1, 68.7, 44.9, 41.9, 30.1, 22.3, 17.5.

### 2-Methoxy-2-phenylethanol (3v)

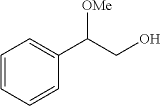 [SMILES: COC(CO)c1ccccc1]

The compound was prepared as described in the general procedure (light yellow liquid, mass=141 mg, 93% yield); ¹H NMR (400 MHz, CDCl₃) δ 7.39-7.28 (m, 5H), 4.31 (dd, J=8.4, 3.8 Hz, 1H), 3.69-3.58 (m, 2H), 3.31 (s, 3H), 2.20 (s, 1H). ¹³C{H} NMR (101 MHz, CDCl₃) δ 138.2, 128.5, 128.1, 126.8, 84.5, 67.3, 56.8.

### 2-Phenoxyethan-1-ol (3w)

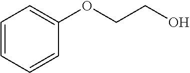

The compound was prepared as described in the general procedure (colorless oil, mass=133 mg, 96% yield); ¹H NMR (400 MHz, CDCl₃) δ 7.34-7.23 (m, 2H), 7.01-6.93 (m, 1H), 6.96-6.89 (m, 2H), 4.10-4.06 (m, 2H), 3.98-3.94 (m, 2H), 2.07 (s, 1H). ¹³C{H} NMR (101 MHz, CDCl₃) δ 158.5, 129.4, 121.1, 114.5, 69.0, 61.4.

### 1-Phenylethane-1,2-diol (3x)

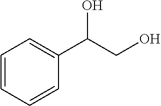

The compound was prepared as described in the general procedure (white solid, mass=101 mg, 73% yield); ¹H NMR (400 MHz, CDCl₃) δ 7.38-7.23 (m, 5H), 4.77 (dd, J=8.3, 3.4 Hz, 1H), 3.70 (dd, J=11.4, 3.5 Hz, 1H), 3.62 (dd, J=11.4, 8.4 Hz, 1H), 3.48 (s, 1H), 3.09 (s, 1H). ¹³C{H}NMR (101 MHz, CDCl₃) δ 140.4, 128.4, 127.9, 126.0, 74.6, 67.9.

### 1-Phenylethane-1,2-diol (3y)

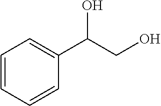

The compound was prepared as described in the general procedure (white solid, mass=55 mg, 40% yield); Compound characterization matches with the compound (3x).

### Cinmmayl Alcohol (3z)

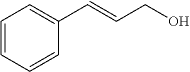

The compound was prepared as described in the general procedure (crystal, mass=54 mg, 40% yield); ¹H NMR (400 MHz, CDCl₃) δ 7.39 (d, J=8.6 Hz, 2H), 7.32 (t, J=7.4 Hz, 2H), 7.29-7.21 (m, OH), 6.61 (d, J=15.9 Hz, 1H), 6.36 (dt, J=14.5, 5.0 Hz, 1H), 4.32 (d, J=5.8 Hz, 2H), 1.91 (s, 1H). ¹³C{H} NMR (101 MHz, CDCl₃) δ 136.6, 131.0, 128.5, 128.4, 127.6, 126.4, 63.6.

### Pentan-1-ol (3aa)

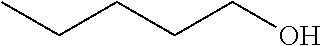

The compound was prepared as described in the general procedure (product not completely isolated due to low boiling point, NMR ratio of pentanol to pent-2-enol, 100:0); ¹H NMR (400 MHz, CDCl₃) δ 3.75 (t, J=6.7 Hz, 2H), 1.63-1.46 (m, 3H), 1.32 (tdd, J=6.2, 2.6, 1.4 Hz, 4H), 0.91-0.87 (m, 3H). ¹³C{H} NMR (101 MHz, CDCl₃) δ 63.1, 31.2, 27.9, 22.4, 14.0.

### 10-Undecylen-1-ol (3ab)

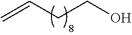

The compound was prepared as described in the general procedure (colorless oil=126 mg, 74% yield); ¹H NMR (400 MHz, CDCl₃) δ 5.79 (ddt, J=16.9, 10.0, 6.7 Hz, 1H), 5.02-4.88 (m, 2H), 3.61 (t, J=6.7 Hz, 2H), 2.02 (quint., J=7.1 Hz, 2H), 1.54 (p, J=7.1 Hz, 2H), 1.40-1.24 (m, 13H). ¹³C{H} NMR (101 MHz, CDCl₃) δ 139.1, 114.0, 62.9, 33.7, 32.7, 29.4, 29.3, 29.0, 28.8, 25.6.

### Hexan-1-ol (3ac)

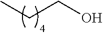

The compound was prepared as described in the general procedure (colorless oil, mass=123 mg, 84% yield); ¹H NMR (400 MHz, CDCl₃) δ 3.63 (td, J=6.7, 1.0 Hz, 2H), 1.61-1.50 (m, 3H), 1.38-1.25 (m, 6H), 0.88 (t, J=6.7 Hz, 3H). ¹³C{H} NMR (101 MHz, CDCl₃) δ 63.0, 32.7, 31.5, 25.3, 22.5, 13.9.

### Pentadecan-1-ol (3ad)

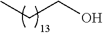

The compound was prepared as described in the general procedure (beige oil, mass=169 mg, 74% yield); ¹H NMR (400 MHz, CDCl₃) δ 3.64 (t, J=6.8 Hz, 2H), 1.60-1.52 (m, 3H), 1.25 (s, 24H), 0.88 (t, J=6.6 Hz, 3H). ¹³C{H} NMR (101 MHz, CDCl₃) δ 63.0, 32.7, 31.8, 29.6, 29.6, 29.5, 29.3, 29.3, 25.6, 22.6, 14.0.

### Cyclohexylmethanol (3ae)

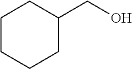

The compound was prepared as described in the general procedure (pale yellow oil, mass=82 mg, 72% yield); ¹H NMR (400 MHz, CDCl₃) δ 3.44 (d, J=6.1 Hz, 2H), 1.79-1.63 (m, 5H), 1.50-1.35 (m, 2H), 1.29-1.09 (m, 3H), 0.93 (q, J=11.3 Hz, 2H). ¹³C{H} NMR (101 MHz, CDCl₃) δ 68.7, 40.4, 29.5, 26.5, 25.7.

### 2-ethyl-2-methylbutan-1-ol (3af)

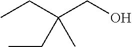

The compound was prepared as described in the general procedure (colorless oil, mass=73 mg, 63% yield); ¹H NMR (400 MHz, CDCl₃) δ 3.34 (s, 1H), 1.27 (qd, J=7.5, 2.8 Hz, 5H), 0.83 (s, 1H), 0.81-0.78 (m, 8H). ¹³C{H} NMR (101 MHz, CDCl₃) δ 68.9, 37.2, 28.0, 20.7, 7.7.

### 2-Bromo-1-dodecanol (3ag)

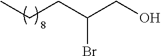

The compound was prepared as described in the general procedure (colorless oil, mass=212 mg, 80% yield; ¹H NMR (400 MHz, CDCl₃) δ 4.19-4.08 (m, 1H), 3.81 (dd, J=12.3, 3.9 Hz, 1H), 3.73 (dd, J=12.3, 6.9 Hz, 1H), 2.05 (s, 1H), 1.83 (q, J=6.7 Hz, 2H), 1.25 (s, 16H), 0.87 (t, J=6.7 Hz, 3H). ¹³C{H} NMR (101 MHz, CDCl₃) δ 67.2, 60.1, 34.8, 31.8, 29.5, 29.4, 29.3, 29.2, 28.9, 27.4, 22.6, 14.0.

### 1,12-Dodecanediol (3ah)

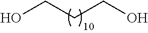

The compound was prepared as described in the general procedure (white solid, mass=147 mg, 73% yield); ¹H NMR (400 MHz, CDCl₃) δ 3.63 (t, J=6.6 Hz, 4H), 1.60-1.50 (m, 4H), 1.35-1.25 (m, 16H). ¹³C{H} NMR (101 MHz, CDCl₃) δ 63.0, 32.7, 29.5, 29.4, 29.3, 25.6.

### Hexane-1,6-diol (3ai)

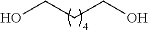

The compound was prepared as described in the general procedure (colorless oil, mass=65 mg, 55% yield); ¹H NMR (400 MHz, CDCl₃) δ 3.60 (t, J=6.6 Hz, 4H), 2.31 (s, 2H), 1.59-1.48 (m, 4H), 1.40-1.32 (m, 4H). ¹³C{H} NMR (101 MHz, CDCl₃) δ 62.5, 32.5, 25.4.

### (2-aminophenyl)methanol (3aj)

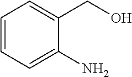

The compound was prepared as described in the general procedure (white solid, mass=47 mg, 38% yield); ¹H NMR (400 MHz, CDCl₃) δ 7.13 (td, J=7.7, 1.6 Hz, 1H), 7.09-7.03 (m, 1H), 6.76-6.66 (m, 2H), 4.65 (s, 2H), 3.58 (s, 3H). ¹³C{H} NMR (101 MHz, CDCl₃) δ 145.9, 129.3, 129.1, 124.7, 118.1, 115.9, 64.3.

### (R)-2-amino-2-phenylethan-1-ol (3ak)

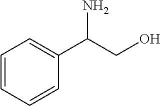

The compound was prepared as described in the general procedure (colorless oil, mass=30 mg, 22% yield); ¹H NMR (400 MHz, CDCl₃) δ 7.35-7.23 (m, 5H), 4.01 (dd, J=8.3, 4.2 Hz, 1H), 3.70 (dd, J=10.8, 4.2 Hz, 1H), 3.53 (dd, J=10.8, 8.3 Hz, 1H), 2.58 (s, 3H). ¹³C{H} NMR (101 MHz, CDCl₃) δ 142.5, 128.5, 127.4, 126.5, 67.8, 57.3.

### tert-butyl (R)-(2-hydroxy-1-phenylethyl)carbamate (3al)

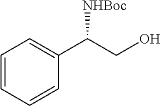 [SMILES: CC(C)(C)OC(=O)N[C@H](CO)c1ccccc1]

The compound was prepared as described in the general procedure (white solid, mass=108 mg, 92% yield); ¹H NMR (400 MHz, CDCl₃) δ 7.37-7.29 (m, 2H), 7.30-7.22 (m, 3H), 5.40 (s, 1H), 4.74 (s, 1H), 3.77 (s, 2H), 2.92 (s, 1H), 1.42 (s, 9H). ¹³C{H} NMR (101 MHz, CDCl₃) δ 156.1, 139.5, 128.6, 127.5, 126.5, 79.8, 66.5, 56.7, 28.3.

### tert-butyl (2-hydroxyethyl)carbamate (3am)

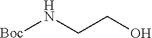 [SMILES: CC(C)(C)OC(=O)NCCO]

The compound was prepared as described in the general procedure (colorless oil, mass=135 mg, 84% yield), in 20 mmol scale reaction using MTBE as solvent and 15 mol % TiCl₄ (colorless oil, mass=2.88 g, 89% yield); ¹H NMR (400 MHz, CDCl₃) δ 4.92 (s, 1H), 3.70 (t, J=5.0 Hz, 2H), 3.29 (q, J=5.2 Hz, 2H), 2.27 (s, 1H), 1.45 (s, 9H). ¹³C{H} NMR (101 MHz, CDCl₃) δ 156.7, 79.4, 61.9, 42.9, 28.2.

### 1-Boc-2-piperidinemethanol (3an)

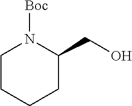 [SMILES: CC(C)(C)OC(=O)N1CCCC[C@@H]1CO]

The compound was prepared as described in the general procedure (colorless oil, mass=201 mg, 93% yield); ¹H NMR (400 MHz, CDCl₃) δ 4.27-4.17 (m, 1H), 3.88 (d, J=13.7 Hz, 1H), 3.71 (dd, J=11.0, 8.5 Hz, 1H), 3.55 (dd, J=11.0, 6.5 Hz, 1H), 2.84-2.71 (m, 2H), 1.71-1.62 (m, 1H), 1.58-1.48 (m, 3H), 1.47-1.31 (m, 11H). ¹³C{H} NMR (101 MHz, CDCl₃) δ 156.0, 79.6, 61.1, 52.2, 39.9, 28.3, 25.1, 24.9, 19.4.

### (9H-fluoren-9-yl)methyl (2-hydroxyethyl)carbamate (3ao)

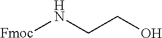

The compound was prepared as described in the general procedure (white solid, mass=218 mg, 77% yield); ¹H NMR (400 MHz, CDCl₃) δ 7.76 (d, J=7.6 Hz, 2H), 7.59 (d, J=7.4 Hz, 2H), 7.40 (t, J=7.3 Hz, 2H), 7.31 (td, J=7.5, 1.2 Hz, 2H), 5.18 (s, 1H), 4.43 (d, J=6.8 Hz, 2H), 4.21 (t, J=6.8 Hz, 1H), 3.71 (t, J=5.0 Hz, 2H), 3.34 (d, J=5.3 Hz, 2H), 2.14 (s, 1H). ¹³C{H} NMR (101 MHz, CDCl₃) δ 157.0, 143.8, 141.2, 127.6, 127.0, 124.9, 119.9, 66.7, 62.2, 47.2, 43.4.

### (9H-fluoren-9-yl)methyl (1-hydroxypropan-2-yl)carbamate (3ap)

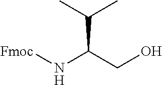

The compound was prepared as described in the general procedure (white solid, mass=240 mg, 74% yield); ¹H NMR (400 MHz, CDCl₃) δ 7.75 (d, J=7.6 Hz, 2H), 7.59 (d, J=7.5 Hz, 2H), 7.39 (t, J=7.5 Hz, 2H), 7.30 (t, J=7.4 Hz, 2H), 5.03 (d, J=9.1 Hz, 1H), 4.43 (dt, J=32.4, 10.4 Hz, 2H), 4.21 (t, J=6.8 Hz, 1H), 3.73-3.57 (m, 2H), 3.48 (s, 1H), 2.50 (s, 1H), 1.92-1.79 (m, 1H), 0.93 (dd, J=12.1, 6.8 Hz, 6H). ¹³C{H} NMR (101 MHz, CDCl₃) δ 157.0, 143.8, 141.3, 127.6, 127.0, 124.9, 119.9, 66.5, 63.5, 58.5, 47.3, 29.1, 19.4, 18.5.

### Procedure for Competitive Reactions:

A 50 mL oven dried round bottom flask was charged with carboxylic acid (1 mmol, 1 eq) and a magnetic stir bar. Either ketone (1 mmol, 1 eq), ester (1 mmol, 1 eq), amide (1 mmol, 1 eq), or nitrile (1 mmol, 1 eq) was then also added. The flask was sealed using a rubber septum. After purging the flask with nitrogen, distilled diethyl ether (3 mL) was added, and the solution was cooled at 0° C. with an ice bath. Subsequently, titanium tetrachloride (TiCl₄) (0.1 mmol, 0.1 eq) was added dropwise to the solution through the septum using a syringe. The septum was then carefully opened and solid ammonia borane (NH₃—BH₃) (2.0 mmol, 2.0 eq) was added slowly to the reaction mixture. Upon complete addition, the reaction flask was again sealed with a septum. After stirring at 0° C. for 1 minute, the reaction mixture was allowed to warm up to room temperature and stirred for 4 hours. On completion of the reaction, the crude mixture was brought to 0° C. and quenched by slow addition of cold water, then transferred to a separatory funnel and extracted with diethyl ether (2×3 mL). The combined organic layers were washed with brine (1×3 mL), dried over anhydrous sodium sulfate, filtered through cotton, and concentrated under rotary evaporation, and the remaining solvent was removed by applying high vacuum for 30 minutes. The resulting crude mixture was analyzed by ¹H NMR to determine the product/starting material ratios.

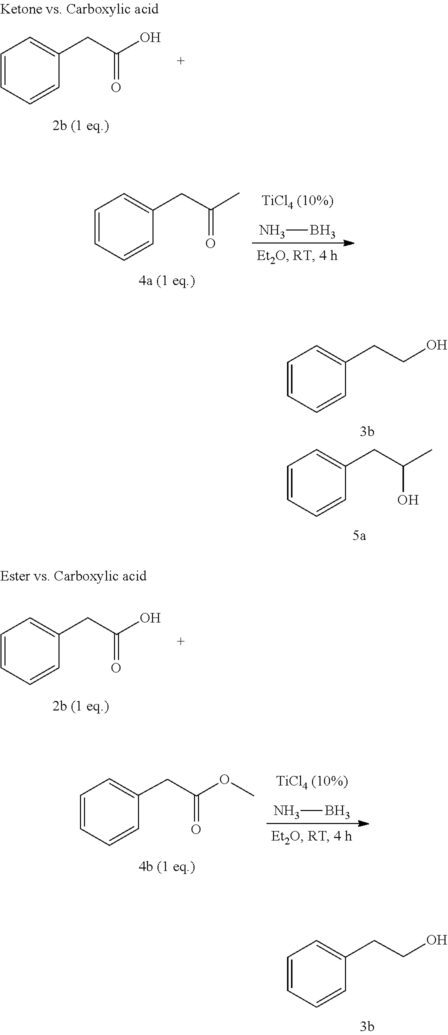

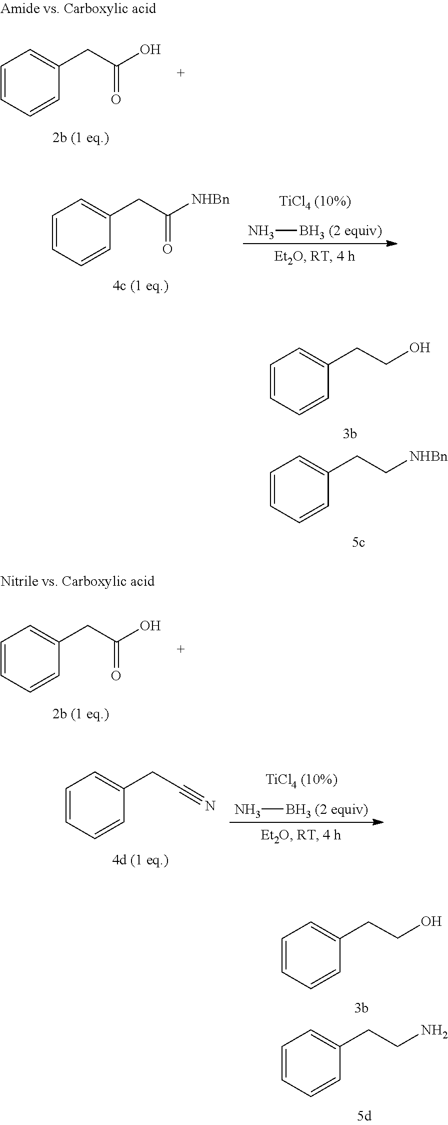

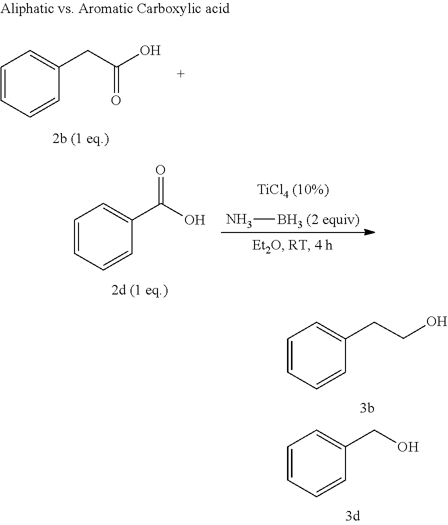

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

The term “substituted” (e.g., as in “optionally substituted”) refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, aralkyloxy groups, oxo(carbonyl) groups, and carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” refers to substituted or unsubstituted straight chain and branched alkyl groups and cycloalkyl groups having from 1 to about 20 carbon atoms (e.g., C₁-C₂₀), 1 to 12 carbons (e.g., C₁-C₁₂), 1 to 8 carbon atoms (e.g., C₁-C₈), or, in some embodiments, from 1 to 6 carbon atoms (e.g., C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” refers to substituted or unsubstituted straight chain and branched divalent alkenyl and cycloalkenyl groups having from 2 to 20 carbon atoms (e.g., C₂-C₂₀), 2 to 12 carbons (e.g., C₂-C₁₂), 2 to 8 carbon atoms (e.g., C₂-C₈) or, in some embodiments, from 2 to 4 carbon atoms (e.g., C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

An alkynyl group is a substituent, which contains an open point of attachment on a carbon atom that would form if a hydrogen atom bonded to a triply bonded carbon is removed from the molecule of an alkyne. The term “hydroxyalkyl” refers to alkyl groups as defined herein and substituted with at least one hydroxyl (—OH) group.

The term “cycloalkyl” refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (e.g., C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and cryloyl groups and the like. When the group containing the carbon atom that is bonded to the carbonyl carbon atom contains a halogen, the group is termed a “haloacyl” group. An example is a trifluoroacetyl group.

The term “aryl” refers to substituted or unsubstituted cyclic aromatic hydrocarbons that do not contain heteroatoms in the ring. Thus, aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (e.g., C₆-C₁₄) or from 6 to 10 carbon atoms (e.g., C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “heterocyclyl” refers to substituted or unsubstituted aromatic and non-aromatic ring compounds containing three or more ring members, of which one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups can include 3 to 8 carbon atoms (e.g., C₃-C₈), 3 to 6 carbon atoms (e.g., C₃-C₆) or 6 to 8 carbon atoms (e.g., C₆-C₈). A heteroaryl ring is an embodiment of a heterocyclyl group. The phrase “heterocyclyl group” includes fused ring species including those that include fused aromatic and non-aromatic groups. Representative heterocyclyl groups include, but are not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” refers to alkyl groups as defined herein in which a hydrogen or carbon bond of an alkyl group is replaced with a bond to a heteroaryl group as defined herein.

The term “alkoxy” refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

It is understood that each of alkyl, cycloalkyl, alkenyl, cycloalkenyl, alkylene, and heterocyclyl, aryl, heteroarylalkyl, heterocyclylalkyl may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialky-lamino, acylamino, thio, and the like, and combinations thereof.

The term “amine” refers to primary, secondary, and tertiary amines having, e.g., the formula N(group)3 wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” refers to a substituent of the form —NH₂, —NHR, —NR₂, —NR₃+, wherein each R is independently selected, and protonated forms of each, except for —NR₃+, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” and “halide” group, by themselves or as part of another substituent, mean, unless otherwise stated, a fluorine, chlorine, bromine, or iodine atom. The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH3)2 and the like.

The terms “optionally substituted” and “optional substituents” are used to describe groups, which are either unsubstituted or substituted with one or more of the substituents specified. When the groups in question are substituted with more than one substituent, the substituents can be the same or different. The terms “independently” “independently are” and “independently selected from” mean that the groups in question may be the same or different. Certain of the defined groups or substituents can occur more than once in the structure, and upon such occurrence each group or substituent shall be defined independently of the other.

The compounds may contain one or more chiral centers, or may otherwise be capable of existing as multiple stereoisomers. In various embodiments, the compounds are not limited to any particular stereochemical requirement, and the compounds, and compositions, methods, uses, and medicaments that include them, may be optically pure or any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. In various embodiments, the compounds are not limited to any particular geometric isomer requirement, and the compounds, and compositions, methods, uses, and medicaments that include them, may be pure or any of a variety of geometric isomer mixtures. Such mixtures of geometric isomers may include a single configuration at one or more double bonds, while including mixtures of geometry at one or more other double bonds.

Those skilled in the art will appreciate that the invention described herein is subject to variations and modifications other than those specifically described herein. It is to be understood that the invention includes all such variations and modifications. The invention also includes all of the steps, features, compounds, and compositions referred to or indicated in this specification, individually or collectively, and any combinations of any two or more of said steps or features.

While the present invention is described herein with reference to illustrated embodiments, it should be understood that the invention is not limited to these examples. Therefore, the present invention is limited by the claims attached herein.

We claim:

1. A process for reducing a carboxylic acid to an alcohol, which process comprises reacting a carboxylic acid with an amine borane in the presence of a Lewis acid catalyst and a solvent.

2. The process of claim 1, wherein the amine borane is represented by formula (I):
RR′R″N→BH₃ (I)
wherein each R, R′, and R″ independently is alkyl, cycloalkyl, heteroalkyl, heterocycloalkyl, aryl, heteroaryl, alkylaryl, or arylalkyl, wherein each R, R′, and R″ is optionally independently substituted with at least one of halo, OH, OR, CONR, alkyl, cycloalkyl, heteroalkyl, heterocycloalkyl, aryl, heteroaryl, alkylaryl, and arylalkyl; or
R, R′ and R″ together, with the N atom to which they are attached, form a 4- to 8-membered heteroaryl ring through an alkylene group; or
R and R′ together, with the N atom to which they are attached, form a 4- to 8-membered heterocyclic ring, which contains one or more heteroatoms.

3. The process of claim 2, wherein the amine-borane is:

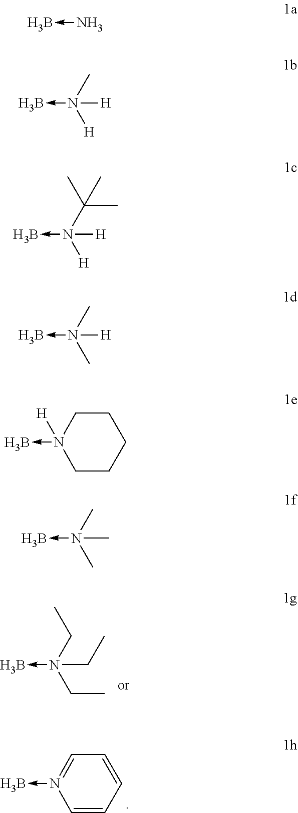

4. The process of claim 1, wherein the amine-borane is an ammonia borane.

5. The process of claim 1, wherein the carboxylic acid is represented by formula (II): 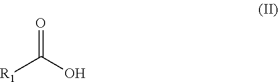
wherein R₁ is selected from the group consisting of alkyl, cycloalkyl, heteroalkyl, heterocycloalkyl, aryl, heteroaryl, alkylaryl, and arylalkyl, wherein R₁ is optionally substituted with at least one of halo, OH, OR₁, CONR₁, alkyl, cycloalkyl, heteroalkyl, heterocycloalkyl, aryl, heteroaryl, alkylaryl, and arylalkyl.

6. The process of claim 1, wherein the alcohol is represented by formula (III): 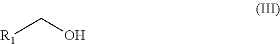
wherein R₁ is selected from the group consisting of alkyl, cycloalkyl, heteroalkyl, heterocycloalkyl, aryl, heteroaryl, alkylaryl, and arylalkyl, wherein R₁ is optionally substituted with at least one of halo, OH, OR₁, CONR₁, alkyl, cycloalkyl, heteroalkyl, heterocycloalkyl, aryl, heteroaryl, alkylaryl, and arylalkyl.

7. The process of claim 1, wherein the Lewis acid catalyst is titanium tetrabromide (TiBr₄), hafnium tetrachloride (HfCl₄), boron trifluoride etherate (BF₃·OEt₂O), aluminium chloride (AlCl₃), bismuth chloride (BiCl₃), ferric chloride (FeCl₃), indium chloride (InCl₃), copper chloride (CuCl₂), zirconium chloride (ZrCl₄), zinc chloride (ZnCl₂), scandium triflate (Sc(OTf)₃), tin chloride (SnCl₂), tin tetrachloride (SnCl₄), or titanium tetrachloride (TiCl₄).

8. The process of claim 7, wherein the Lewis acid catalyst is TiCl₄.

9. The process of claim 1, wherein the Lewis acid is used in an amount of about 0.01 mol % to about 100 mol % relative to an amount of carboxylic acid.

10. The process of claim 9, wherein the amount of Lewis acid used is about 0.05 mol % to about 10 mol % relative to the amount of carboxylic acid.

11. The process of claim 1, wherein the amine-borane is used in an amount based on the amount of carboxylic acid from about 100 mol % to about 200 mol %.

12. The process of claim 11, wherein the amount of amine-borane used based on the amount of carboxylic acid is 100 mol %.

13. The process of claim 1, wherein the solvent used is an organic solvent.

14. The process of claim 13, wherein the organic solvent is selected from diethyl ether, tetrahydrofuran, dichloromethane, 2-methyl tetrahydrofuran, 1,2-dimethoxyethane, methyl tert-butyl ether, and acetonitrile.

15. The process of claim 1, wherein the Lewis acid catalyst is added into a solution of the carboxylic acid prior to an addition of the amine-borane.

16. The process of claim 1, wherein the process is carried out at a temperature from about 0° C. to 150° C.

17. The process of claim 1, wherein the process is carried out in a batch process or in a continuous process.
